(19) United States
(12) Patent Application Publication
(10) Pub. No.: US 2010/0159037 A1
(43) Pub. Date: Jun. 24, 2010

(54) Vitamin Breath Freshener

(76) Inventor: Yolanda Eustaquio, Miami, FL (US)

Correspondence Address: RUBEN ALCOBA, ESQ., 3399 NW 72 AVENUE, SUITE211, MIAMI, FL, 33122, US

(21) Appl. No.: 12/340,969

(22) Filed: Dec. 22, 2008

(51) Int. Cl. A61K 36/534; A61P 1/02

(52) U.S. Cl. 424/747

(57) ABSTRACT

A liquid breath spray for orally delivering a vitamin dosage and freshening the mouth of a user.

### BACKGROUND

The present invention relates to the fields of oral hygiene and oral vitamin delivery.

One of the most troubling aspects of society today is that very few people have or take the time to care for their health. Unfortunately, living a healthy lifestyle has taken a backseat compared to vanity, for most people. For example, if you look into most peoples' medicine cabinet, you will probably find a bottle of mouthwash, yet you will not find one vitamin supplement.

This dilemma caused the present inventor to conceive a product that would appeal to ones vanity while at the same time delivering a health supplement. She realized that most people were vitamin deficient because of the hustle and flow of the modern times.

She realized that she had to develop a product that would be used by people to deliver a vitamin that would not take them out of their routine. People tend to care greatly about their oral hygiene, for it is one of the first aspects a mate will have to consider when first meeting a prospective partner.

An object of the present invention is to provide a disposable oral delivery vitamin shot.

Another object of the present invention is to provide a device that will allow a user to freshen his or her breath while concurrently absorbing a vitamin supplement.

A further object of the present invention is to provide users with a product that will fulfill two purposes, breath freshening and vitamin supplementation.

### SUMMARY

The present invention is directed to a product that is both a breath freshener and a vitamin supplement. The product is a liquid breath spray that orally delivers a vitamin dosage while freshening the mouth of the user. The liquid breath freshener satisfies the need of having a product that will serve two purposes: it is a vitamin supplement and a breath freshener.

These and other features, aspects and advantages of the present invention will become better understood with reference to the following description and appended claims.

### DESCRIPTION

A liquid breath spray for orally delivering a vitamin dosage and freshening the mouth of a user, comprising a sprayable liquid consisting essentially of: a customized blend of vitamins in the amount of approximately 10.0 percent by volume; essential mint oil in the amount of approximately 10.0 percent by volume; propylene glycol in the range from about approximately 0.1 percent to about approximately 0.5 percent by volume; sodium benzoate in the amount of approximately 0.5 percent by volume; potassium sorbate in the amount of approximately 1.0 percent by volume; glucose in the range from about approximately 1.0 percent to about approximately 12.0 by volume; and water.

In an embodiment of the present invention, the customized blend of vitamins might be comprised of vitamins B1, B2, B3, B5, B6, B12 and C. It is foreseen that other vitamins could comprise the customized blend.

In another embodiment of the present invention, the liquid breath spray might be stored in a 15 ml dosage container. The 15 ml dosage container provides the user with 20 percent of the recommended daily allowance of each vitamin contained in the container.

The liquid breath spray is made as follows: 1. diluting the vitamin mixture into the water, thereby creating a first mixture; 2. diluting the essential mint into the propylene glycol, thereby creating a second mixture; 3 mixing the first mixture and the second mixture, thereby creating a third mixture; 4. adding the potassium sorbate and the glucose to the third mixture, thereby creating a fourth mixture; 5. adding sodium benzoate to the fourth mixture, thereby creating a fifth mixture; 6. heating the fifth mixture at 100 degrees Fahrenheit until the sodium benzoate is completely dissolved; and 7. cooling the fifth mixture for at least 25 minutes.

Essential mint oil is used to provide the breath freshener quality to the product. It is foreseen that similar oils could be used to provide the breath freshening quality to the product.

An advantage of the present invention is that it provides a disposable oral delivery vitamin shot.

Another advantage of the present invention is that it provides a device that allows a user to freshen his or her breath while concurrently absorbing a vitamin supplement.

A further advantage of the present invention is that it provides users with a product that fulfills two purposes, breath freshening and vitamin supplementation.

While we have shown and described embodiments in accordance with the present invention, it should be clear to those skilled in the art that further embodiments may be made without departing from the scope of the present invention.

What is claimed is:

1. A liquid breath spray for orally delivering a vitamin dosage and freshening the mouth of a user, comprising a sprayable liquid consisting essentially of:
a customized blend of vitamins in the amount of approximately 10.0 percent by volume;
essential mint oil in the amount of approximately 10.0 percent by volume;
propylene glycol in the range from about approximately 0.1 percent to about approximately 0.5 percent by volume;
sodium benzoate in the amount of approximately 0.5 percent by volume;
potassium sorbate in the amount of approximately 1.0 percent by volume;
glucose in the range from about approximately 1.0 percent to about approximately 12.0 by volume; and
water.

2. The liquid breath spray of claim 1, wherein the customized blend of vitamins is comprised of vitamins B1, B2, B3, B5, B6, B12 and C.

3. The liquid breath spray of claim 2, wherein the spray is stored in a 15 ml dosage container.

4. The liquid breath spray of claim 3, wherein the dosage is consumed within a 24 hour period.

5. A method of making the liquid breath spray of claim 1, comprising the steps of:
diluting the vitamin mixture into the water, thereby creating a first mixture;
diluting the essential mint into the propylene glycol, thereby creating a second mixture;
then, mixing the first mixture and the second mixture, thereby creating a third mixture;
next, adding the potassium sorbate and the glucose to the third mixture, thereby creating a fourth mixture;
then, adding sodium benzoate to the fourth mixture, thereby creating a fifth mixture;
next heating the fifth mixture at 100 degrees Fahrenheit until the sodium benzoate is completely dissolved; and
lastly, cooling the fifth mixture for at least 25 minutes.
